SEQUENCE LISTING (28 sequences)

SEQ ID NO: 1 - DNA, 19 bp, Artificial
  Synthetic PCR Primer
ggtgcgctgtctgtctgag

SEQ ID NO: 2 - DNA, 18 bp, Artificial
  Synthetic PCR Primer
ctgatctcgccgttgagg

SEQ ID NO: 3 - DNA, 20 bp, Artificial
  Synthetic PCR Primer
gcagcacctcatcgactaca

SEQ ID NO: 4 - DNA, 20 bp, Artificial
  Synthetic PCR Primer
aattcagaagcctgcaagga

SEQ ID NO: 5 - DNA, 20 bp, Artificial
  Synthetic PCR Primer
ctggacgacatgaaccactg

SEQ ID NO: 6 - DNA, 20 bp, Artificial
  Synthetic PCR Primer
gtagtcgatgacgcgctgta

SEQ ID NO: 7 - DNA, 25 bp, Artificial
  Synthetic PCR Primer
atgaaggcggtgagcccggtgcgcc

SEQ ID NO: 8 - DNA, 25 bp, Artificial
  Synthetic PCR Primer
tgtggccgtgctcggccaggcagcg

SEQ ID NO: 9 - DNA, 20 bp, Artificial
  Synthetic PCR Primer
gagtccactggcgtcttcac

SEQ ID NO: 10 - DNA, 20 bp, Artificial
  Synthetic PCR Primer
ctcagtgtagcccaggatgc

SEQ ID NO: 11 - DNA, 21 bp, Artificial
  Synthetic PCR Primer
gggaaggtattcagccaaacg

SEQ ID NO: 12 - DNA, 20 bp, Artificial
  Synthetic PCR Primer
ggttcgctttctctttcggg

SEQ ID NO: 13 - DNA, 23 bp, Artificial
  Synthetic PCR Primer
aatacctcagcctccagcagatg

SEQ ID NO: 14 - DNA, 22 bp, Artificial
  Synthetic PCR Primer
caaagcagcctccaagtcactg

SEQ ID NO: 15 - DNA, 22 bp, Artificial
  Synthetic PCR Primer
cctggaggaatacctggcattg

SEQ ID NO: 16 - DNA, 20 bp, Artificial
  Synthetic PCR Primer
tctgaggacaagcgattgcg

SEQ ID NO: 17 - DNA, 24 bp, Artificial
  Synthetic PCR Primer
tgagatcacttcaccgtggtctcc

SEQ ID NO: 18 - DNA, 22 bp, Artificial
  Synthetic PCR Primer
tttatacctcggggttgtgggg

SEQ ID NO: 19 - DNA, 23 bp, Artificial
  Synthetic PCR Primer
cgtccatctttgcttgggaaatc

SEQ ID NO: 20 - DNA, 23 bp, Artificial
  Synthetic PCR Primer
gagcctcatctgaatcttctccg

SEQ ID NO: 21 - DNA, 24 bp, Artificial
  Synthetic PCR Primer
aagccatgaacgcagaggaggact

SEQ ID NO: 22 - DNA, 20 bp, Artificial
  Synthetic PCR Primer
agctgtccatggtaccgtaa

SEQ ID NO: 23 - DNA, 24 bp, Artificial
  Synthetic PCR Primer
aacccaactgtggagatgatgcag

SEQ ID NO: 24 - DNA, 24 bp, Artificial
  Synthetic PCR Primer
aggggcttcactaataactggacg

SEQ ID NO: 25 - DNA, 21 bp, Artificial
  Synthetic PCR Primer
ccaagccgccttactcctaca

SEQ ID NO: 26 - DNA, 21 bp, Artificial
  Synthetic PCR Primer
cgcagatgaagacgctggaga

SEQ ID NO: 27 - DNA, 25 bp, Artificial
  Synthetic PCR Primer
tggcaccacaccttctacaatgagc

SEQ ID NO: 28 - DNA, 25 bp, Artificial
  Synthetic PCR Primer
gcacagcttctccttaatgtcacgc

(19) United States
(12) Patent Application Publication
(10) Pub. No.: US 2010/0173410 A1
(43) Pub. Date: Jul. 8, 2010

(54) Cultivation of Primate Embryonic Stem Cells

(75) Inventors: James A. Thomson, Madison, WI (US); Mark Levenstein, Madison, WI (US); Ren-He Xu, Farmington, CT (US)

Correspondence Address: QUARLES & BRADY LLP, 411 E. WISCONSIN AVENUE, SUITE 2040, MILWAUKEE, WI, 53202-4497, US

(73) Assignee: WiCell Research Institute, Inc., Madison, WI (US)

(21) Appl. No.: 12/489,978

(22) Filed: Jun. 23, 2009

Related U.S. Application Data
(63) Continuation-in-part of application No. 12/240,657, filed on Sep. 29, 2008, now abandoned.
Application No. 12/240,657 is a continuation of application No. 11/134,564, filed on May 20, 2005, now Pat. No. 7,514,260.
Application No. 11/134,564 is a continuation-in-part of application No. 12/240,640, filed on Sep. 29, 2008, now abandoned.
Application No. 12/240,640 is a continuation of application No. 11/078,737, filed on Mar. 11, 2005, now Pat. No. 7,439,064.
Application No. 11/078,737 is a continuation-in-part of application No. 10/952,096, filed on Sep. 28, 2004, now abandoned.
Application No. 10/952,096 is a continuation-in-part of application No. 09/522,030, filed on Mar. 9, 2000, now Pat. No. 7,005,252.
(60) Provisional application No. 60/573,545, filed on May 21, 2004.

(51) Int. Cl. C12N 5/0735

(52) U.S. Cl. 435/366

(57) ABSTRACT

The invention relates to methods for culturing human embryonic stem cells by culturing the stem cells in an environment essentially free of mammalian fetal serum and in a stem cell culture medium including amino acids, vitamins, salts, minerals, transferrin, insulin, albumin, and a fibroblast growth factor that is supplied from a source other than just a feeder layer the medium. Also disclosed are compositions capable of supporting the culture and proliferation of human embryonic stem cells without the need for feeder cells or for exposure of the medium to feeder cells.

### CROSS REFERENCES TO RELATED APPLICATIONS

This application is a continuation-in-part of U.S. patent application Ser. No. 12/240,657 and U.S. patent application Ser. No. 12/240,640, both of which were filed Sep. 29, 2008. U.S. patent application Ser. No. 12/240,640 is a continuation of U.S. patent application Ser. No. 11/078,737, filed Mar. 11, 2005 and now U.S. Pat. No. 7,439,064, which is a continuation-in-part of U.S. patent application Ser. No. 10/952,096, filed Sep. 28, 2004, which is a continuation-in-part of U.S. patent application Ser. No. 09/522,030, filed Mar. 9, 2000 and now U.S. Pat. No. 7,005,252. U.S. patent application Ser. No. 12/240,657 is a continuation of U.S. patent application Ser. No. 11/134,564, filed May 20, 2005 and now U.S. Pat. No. 7,514,260, which claims benefit to U.S. Provisional Patent Application 60/573,545 filed May 21, 2004. All applications are incorporated by reference herein.

### STATEMENT REGARDING FEDERALLY SPONSORED RESEARCH

To be determined.

### BACKGROUND OF THE INVENTION

The present invention relates to methods for culturing primate embryonic stem cell cultures and culture media useful therewith.

Primate (e.g. monkey and human) pluripotent embryonic stem cells have been derived from preimplantation embryos. See, for example, U.S. Pat. No. 5,843,780 and J. Thomson et al., 282 Science 1145-1147 (1998). The disclosure of these publications and of all other publications referred to herein are incorporated by reference as if fully set forth herein. Notwithstanding prolonged culture, these cells stably maintain a developmental potential to form advanced derivatives of all three embryonic germ layers.

Primate (particularly human) embryonic stem (ES) cell lines have widespread utility in connection with human developmental biology, drug discovery, drug testing, and transplantation medicine. For example, current knowledge of the post-implantation human embryo is largely based on a limited number of static histological sections. Because of ethical considerations the underlying mechanisms that control the developmental decisions of the early human embryo remain essentially unexplored.

Although the mouse is the mainstay of experimental mammalian developmental biology, and although many of the fundamental mechanisms that control development are conserved between mice and humans, there are significant differences between early mouse and human development. Primate/human ES cells should therefore provide important new insights into their differentiation and function.

Differentiated derivatives of primate ES cells could be used to identify gene targets for new drugs, used to test toxicity or teratogenicity of new compounds, and used for transplantation to replace cell populations in disease. Potential conditions that might be treated by the transplantation of ES cell-derived cells include Parkinson's disease, cardiac infarcts, juvenile-onset diabetes mellitus, and leukemia. See e.g. J. Rossant et al. 17 Nature Biotechnology 23-4 (1999) and J. Gearhart, 282 Science 1061-2 (1998).

Long term proliferative capacity, developmental potential after prolonged culture, and karyotypic stability are key features with respect to the utility of primate embryonic stem cell cultures. Cultures of such cells (especially on fibroblast feeder layers) have typically been supplemented with animal serum (especially fetal bovine serum) to permit the desired proliferation during such culturing.

For example, in U.S. Pat. Nos. 5,453,357, 5,670,372 and 5,690,296 various culture conditions were described, including some using a type of basic fibroblast growth factor together with animal serum. Unfortunately, serum tends to have variable properties from batch to batch, thus affecting culture characteristics.

In WO 98/30679 there was a discussion of providing a serum-free supplement in replacement for animal serum to support the growth of certain embryonic stem cells in culture. The serum replacement included albumins or albumin substitutes, one or more amino acids, one or more vitamins, one or more transferrins or transferrin substitutes, one or more antioxidants, one or more insulins or insulin substitutes, one or more collagen precursors, and one or more trace elements. It was noted that this replacement could be further supplemented with leukemia inhibitory factor, steel factor, or ciliary neurotrophic factor. Unfortunately, in the context of primate embryonic stem cell cultures (especially those grown on fibroblast feeder layers), these culture media did not prove satisfactory.

In the context of nutrient serum culture media (e.g. fetal bovine serum), WO 99/20741 discusses the benefit of use of various growth factors such as bFGF in culturing primate stem cells. However, culture media without nutrient serum are not described.

In U.S. Pat. No. 5,405,772 growth media for hematopoietic cells and bone marrow stromal cells are described. There is a suggestion to use fibroblast growth factor in a serum-deprived media for this purpose. However, conditions for growth of primate embryonic stem cells are not described.

The first human embryonic stem cell cultures were grown using a layer of fibroblast feeder cells, which has the property of enabling the human embryonic stem cells to be proliferated while remaining undifferentiated. Later, it was discovered that it is sufficient to expose the culture medium to feeder cells, to create what is called conditioned medium, which had the same property as using feeder cells directly. Without the use of either feeder cells or conditioned medium, human embryonic stem cells in culture could not be maintained in an undifferentiated state. Since the use of feeder cells, or even the exposure of the medium to feeder cells, risks contamination of the culture with unwanted material, avoiding the use of feeder cells and conditioned medium is desirable. Medium which has not been exposed to feeder cells is referred to here as unconditioned medium.

It can therefore be seen that a need still exists for techniques to stably culture primate embryonic stem cells at a high purity without the requirement for use of animal serum, feeder layer and/or conditioned medium.

### BRIEF SUMMARY OF THE INVENTION

In one aspect the invention provides a method of culturing primate embryonic stem cells. One cultures the stem cells in a culture essentially free of mammalian fetal serum (preferably also essentially free of any animal serum) and in the presence of fibroblast growth factor that is supplied from a source other than a fibroblast feeder layer. In a preferred form, the fibroblast feeder layer, previously required to sustain a stem cell culture, is rendered unnecessary by the addition of sufficient fibroblast growth factor.

Fibroblast growth factors are essential molecules for mammalian development. There are currently more then twenty known fibroblast growth factor ligands and five signaling fibroblast growth factor receptors therefor (and their spliced variants). See generally D. Ornitz et al., 25 J. Biol. Chem. 15292-7 (1996); U.S. Pat. No. 5,453,357. Slight variations in these factors are expected to exist between species, and thus the term fibroblast growth factor is not species limited. However, we prefer to use human fibroblast growth factors, more preferably human basic fibroblast growth factor produced from a recombinant gene. This compound is readily available in quantity from Gibco BRL-Life Technologies and others.

It should be noted that for purposes of this patent the culture may still be essentially free of the specified serum even though a discrete component (e.g. bovine serum albumin) has been isolated from serum and then is exogenously supplied. The point is that when serum itself is added the variability concerns arise. However, when one or more well defined purified component(s) of such serum is added, they do not.

Preferably the primate embryonic stem cells that are cultured using this method are human embryonic stem cells that are true ES cell lines in that they: (i) are capable of indefinite proliferation in vitro in an undifferentiated state; (ii) are capable of differentiation to derivatives of all three embryonic germ layers (endoderm, mesoderm, and ectoderm) even after prolonged culture; and (iii) maintain a normal karyotype (are euploid) throughout prolonged culture. These cells are therefore referred to as being pluripotent.

Preferably, at least 90% of the human embryonic stem cells in the culture retain all the characteristics of human ES cells, including characteristic morphology (small and compact with indistinct cell membranes), proliferation and expression of markers indicative of ES cell status, such as expression of the nuclear transcription factor Oct4.

The culturing permits the embryonic stem cells to stably proliferate in culture for over one month (preferably over six months; even more preferably over twelve months) while maintaining the potential of the stem cells to differentiate into derivatives of endoderm, mesoderm, and ectoderm tissues, and while maintaining the karyotype of the stem cells.

In another aspect the invention provides another method of culturing primate embryonic stem cells. One cultures the stem cells in a culture essentially free of mammalian fetal serum (preferably also essentially free of any animal serum) and in the presence of a growth factor capable of activating a fibroblast growth factor signaling receptor, wherein the growth factor is supplied from a source other than just a fibroblast feeder layer. While the growth factor is preferably a fibroblast growth factor, it might also be other materials such as certain synthetic small peptides (e.g. produced by recombinant DNA variants or mutants) designed to activate fibroblast growth factor receptors. See generally T. Yamaguchi et al., 152 Dev. Biol. 75-88 (1992)(signaling receptors).

In yet another aspect the invention provides a culture system for culturing primate embryonic stem cells. It has a human basic fibroblast growth factor supplied by other than just the fibroblast feeder layer. The culture system is essentially free of animal serum.

Yet another aspect of the invention provides cell lines (preferably cloned cell lines) derived using the above method. “Derived” is used in its broadest sense to cover directly or indirectly derived lines.

Variability in results due to differences in batches of animal serum is thereby avoided. Further, it has been discovered that avoiding use of animal serum while using fibroblast growth factor can increase the efficiency of cloning.

It is therefore an advantage of the present invention to provide culture conditions for primate embryonic stem cell lines where the conditions are less variable and permit more efficient cloning. Other advantages of the present invention will become apparent after study of the specification and claims.

### DETAILED DESCRIPTION OF THE PREFERRED EMBODIMENTS

The observation that human embryonic stem (ES) cell cultures have previously been maintained in an undifferentiated state only when cultured in the presence of fibroblast feeder cells or in conditioned medium has led to speculation that the fibroblasts release into the medium a factor which acts to inhibit differentiation of the ES cells. This speculation is also based on the parallel observations of murine ES cell lines, which, when cultured with fibroblast feeder cells, respond to leukemia inhibitory factor (LIF) secreted by the fibroblasts to remain undifferentiated. The LIF activates a signal pathway in the murine ES cells that triggers self-renewal. However, human ES cells are unresponsive to LIF and indeed do not seem to possess LIF receptors on their cell surface. Since no single factor has been isolated from conditioned medium that seemed to cause the effect of preventing differentiation in human ES cells, we developed a new hypothesis. We hypothesized that instead the fibroblast cells inactivate differentiation factors present in unconditioned medium.

Various research groups have investigated factors that initiate differentiation of human ES cells into progeny cell cultures that are enriched in cells of one or more particular lineage. One of these differentiation factors is a category of protein factor known as bone morphogenetic protein (BMP). BMPs are members of the transforming growth factor-β (TGFβ) superfamily of secreted signaling molecules. They play an extensive role in almost all aspects of embryonic development. BMP 4 and other BMP family members, such as BMP2, -5, and -7, bind BMP type II receptor BRII, which recruits type I receptor BR1A (ALK3) or BR1B. Upon ligand activation, the intracellular kinase domain of the type I receptors phosphorylates Smad1, -5, and -8, which are then escorted by a common Smad to enter the nucleus and activate target genes. The relative expression level of BMPs, receptors, and Smads within the cell is an important determinant of BMP-induced responses. Co-stimulation of other signaling pathways also alters the nature of BMP effect. A typical example is the change of BMP action by a co-activated LIF signal in mouse ES cells: BMP signal alone induces non-neural epithelial differentiation, whereas BMP and LIF signals together inhibit differentiation to any lineage. The extracellular BMP antagonists such as noggin, gremlin, chordin, inhibin, follistatin, twisted gastrulation and members of the DAN family, etc. can modify, diminish or totally nullify BMP activities. On the other hand, some signaling pathways can interrupt the BMP signaling intracellularly. For example, the MAPK signaling activated by fibroblast growth factor (FGF) can inhibit the BMP signaling by preventing the Smads from nuclear translocation via phosphorylation of the linker domain of the Smads. Activation of the transforming growth factor beta (TGFβ), Nodal, or Activin signaling pathways may antagonize the BMP signaling via intracellular cross-talk, such as competition for Smad4 to enter the nucleus. It is anticipated that all of these molecules can be used to antagonize BMP signaling to achieve the effects reported here.

It was also observed that the levels of bone morphogenetic protein (BMP) stimulated intracellular signal is low in human ES cells grown in conditioned medium, whereas the level of this same signal is high in human ES cells grown out in unconditioned medium (and without fibroblast feeder cells). Perhaps the effect of the conditioning of the medium was due to inhibition of the effects of BMP inducing signals present in the unconditioned medium. We therefore explored the possibility that antagonists of BMP activity could act to enable the cultivation of human ES cells in culture and in an undifferentiated state without the need for feeder cells or conditioned medium. It was discovered, and is reported here, that this possibility was found to be correct. By antagonizing the activity of BMP, it has become possible to culture human ES cells indefinitely, while the cells retain all of the identifying characteristics of embryonic stem cells.

There are a number of antagonists of BMP that can be used in this invention. The most potent known such antagonist is the protein noggin. Other proteins known to function as antagonists of BMPs include gremlin, chordin, inhibin, follistatin, twisted gastrulation and members of the DAN family. As mentioned above, other proteins include TFGβ and activin and other molecules which activate the signaling pathway for MAPK. It is not required that the antagonist protein be the human form of the protein. It is only required that it be effective in culture to allow unconditioned medium to maintain ES cells without differentiation. It is also possible to use as an antagonist antibodies specific to all BMPs or a specific BMP. The particular protein chosen as the BMP antagonist is less important than that the desired effect is achieved in that BMP signaling activity is inhibited by the molecule added to the medium. The simplest and most straightforward way to accomplish this is to add the BMP antagonist to the medium in which the human ES cells are cultured.

The most potent BMP inhibitor identified so far, the protein noggin, was originally cloned based on its dorsalizing activity in Xenopus embryos. Mouse noggin cDNA encodes a 232 amino acid (aa) residue precursor protein with 19 aa residue putative signal peptide that is cleaved to generate the 213 aa residue mature protein which is secreted as a homodimeric glycoprotein. Noggin is a highly conserved molecule. Mature mouse noggin shares 99% and 83% aa sequence identity with human and Xenopus noggin, respectively. Noggin has a complex pattern of expression during embryogenesis. In the adult, noggin is expressed in the central nervous system and in several adult peripheral tissues such as lung, skeletal muscle and skin. Noggin has been shown to be a high-affinity BMP binding protein that antagonizes almost all BMP bioactivities.

There appears to be a synergistic relationship between the effect caused by a BMP antagonist and that caused by high levels of FGF in the culture medium. In other words, the use of a high level of bFGF, e.g. at 100 ng/ml, will support cultures of hES cells in an undifferentiated state without feeder cells or conditioned medium, but so will a lesser level of bFGF, e.g. 40 ng/ml when combined with the use of a BMP antagonist such as noggin. Either combination makes the culture not just “feeder free”, which is a term used for cultures which make use of conditioned medium (conditioned with feeder cells) but completely “feeder independent,” meaning entirely independent of the need for feeder cells of any kind at all.

As the data presented below will demonstrate, this hypothesis has proven to be correct. By adding noggin, or other inhibitor of BMP signaling, and by stimulating the fibroblast growth factor (FGF) signal, human ES cells can be grown indefinitely in an undifferentiated state without either feeder cells or conditioned medium. This permits a human ES cell culture to be initiated and maintained without exposure to feeder cells or medium exposed to feeder cells, thus enabling animal cell-free proliferation of human ES cell lines in a well defined medium.

In some of the following experiments one of the inventors here used the methods and culture systems of the invention to culture human ES cell lines without adding serum to the culture

In another of the experiments set forth below, it has now been demonstrated that the addition of relatively large amounts of a human fibroblast growth factor (FGF) aids in the culture and growth of human embryonic stem cells, even in the absence of both serum and feeder cells. This permits the culture of stem cells that have never been exposed either to animal cells or to media in which animal cells have been cultured. These stem cell cultivation conditions (i.e. no feeder cells and no conditioned medium) are referred to here as feeder independent. Prior culture conditions have been described, based on the use of medium conditioned with feeder cells, which are described as feeder free. However, the use of conditioned medium does not resolve the dependence on the use of feeder cells, which still must be used to condition the medium. The techniques described here permit the indefinite and feeder independent culture of human embryonic stem cells having stable karyotype and with the stem cells remaining undifferentiated. Preferably, this technique allows 90% of the stem cells in the culture remain undifferentiated.

Techniques for the initial derivation, culture, and characterization of the human ES cell line H9 were described in J. Thomson et al., 282 Science 1145-1147 (1998). The experiments described below were conducted with this and other cell lines, but the processes and results are independent of any particular ES cells line.

It is described here that the addition of fibroblast growth factor (FGF) aids in the cultivation and cloning of human ES cells. The addition of FGF is important in two distinct regards. First, the addition of FGF at moderate levels (e.g. 4 ng/ml) permits the culture of undifferentiated human ES cells in a medium devoid of serum. At this level, the rate of differentiation of the stem cell is slowed, compared to lower levels of FGF, but the cells will eventually differentiate. Secondly, the addition of FGF at higher levels makes the culture conditions of the medium feeder independent, in that no feeder cells are required at all to indefinitely maintain the pluripotency of euploid undifferentiated human ES cells in culture.

This first phenomenon is believed to be actuated by the action of FGF in interacting with FGF receptors in the human ES cells. To avoid the use of serum, it is not particularly critical which of the many known FGF variants are used in the culture. Here basic FGF, or bFGF, also known as FGF2, is commonly used, but that is only because bFGF is one of the readily commercially available members of the FGF family of factors. More than twenty different FGF family members have been identified, and they are referred to as FGF-1 through FGF-27. While the concentration of FGF here is given in amounts of bFGF, it should be understood that this is intended to quantify the amount of stimulation of the FGF receptors and that the concentration of FGF may have to be adjusted, upward or downward, for other members of the FGF family. For bFGF, the preferred concentration of FGF in the ES cell medium is in the range of about 0.1 to about 1000 ng/ml, with concentrations in excess range of about 4 ng/ml being useful to avoid the need for serum in the medium.

Surprisingly, it has been found that for the second attribute of FGF in a human ES cell medium, the selection of the variant of FGF has some criticality. For this purpose it has been found that when the concentration of bFGF is about 100 ng/ml, this condition is sufficient to avoid the need for both serum and feeder cells, making the culture feeder independent. For this purpose, it has been found that FGF family members FGF2 (bFGF), FGF4, FGF9, FGF17 and FGF18 are each sufficient at 100 ng.ml of culture to make the human ES cell culture feeder independent. By contrast, it has been found that FGF family members FGF1 (acidic FGF), FGF1β, FGF3, FGF5, FGF6, FGF7, FGF8, FGF10, FGF16, FGF19, and FGF 20 are not sufficient at 100 ng/ml to support feeder independence. We believe, but do not have present data, that the results using these forms of FGF are not a result of concentration and that higher concentrations of the particular FGF also would not succeed in supporting feeder independence. For FGF9, our data suggests that at this level (100 ng/ml) FGF9 supports human ES cell culture but the data has been slightly more equivocal.

The exact minimal amount of the effective variants of FGF that will suffice to support human ES cells as feeder independent in culture is not known with precision at this time, but can be determined by empirical testing. It is known that for FGF2, that 4 ng/ml added to the medium alone is insufficient for the indefinite maintenance of euploid undifferentiated human ES cells in culture, while 100 ng/ml of FGF2 alone in the medium is sufficient. While ES cells grown in unconditioned medium containing as little as 4 ng/ml will remain undifferentiated for some time, and perhaps a passage or two, the cells will eventually begin to differentiate. In our hands, the ability of a medium to culture ES cells to remain indefinitely undifferentiated and euploid is demonstrated when the cells are cultured for at least six passages while remaining proliferating, undifferentiated, euploid and while maintaining the characteristic morphology of human ES cells. As used here, a maintenance concentration of an FGF is the concentration of that FGF necessary to support the maintenance of human ES cells in an undifferentiated, euploid and proliferating state for at least six passages. For FGF2, the minimal maintenance concentration is between 4 ng/ml and 100 ng/ml and the exact minimal maintenance concentration can be determined by using the protocols below to interpolate those amounts. For each other effective FGF, e.g. FGF4, FGF9, FGF17, and FGF18, the corresponding minimal maintenance concentration for each FGF can be determined by similar testing.

A related concern in the culture of human ES cells is to remove, to the extent possible, undefined constituents and constituents of animal origin from ES cell culture conditions. This is done for two reasons. One reason is to standardize culture conditions so as to minimize the normal variations in biological materials to the extent possible. The other objective is to avoid the use of materials, cells, exudates or constituents of animal origin so as to avoid any possible cross-species viral transmission through the culture system. Thus it is an objective to define a culture condition that avoids the use of products of animal origin.

So a defined medium for human ES cells begins with a basal medium containing salts, vitamins, glucose and amino acids. The basal medium can be any of a number of commercially available media. We prefer a combination of Dulbecco's Modified Eagle Medium and Hams F12 medium, sold as a combination (DMEM/F12). To that basal medium is added glutamine, β-mercaptoethanol, and non-essential amino acids. Other possible additives include antioxidants and lipids. A protein constituent of the medium is a serum substitute product. Albumin or purified albumin products, like the commercial product AlbuMax™, will work, but we prefer a defined protein product made up of albumin, insulin and transferrin. Human proteins are preferred but not essential so long as uncharacterized animal products are excluded.

Human ES cell cultures in the defined human ES cell media described below in the examples can be cultivated indefinitely in the complete absence of fibroblast feeder cells and without conditioned media while remaining euploid. The ES cells are thus truly feeder independent. The human ES cells retain all of the characteristics of human ES cells including characteristic morphology (small and compact with indistinct cell membranes), proliferation and the ability to differentiate into many, if not all, the cell types in the human body. The human ES cells will also retain the characteristic that they can form all three primordial cell layers when injected into immuno-compromised mice. In particular, the ES cells retain the ability to differentiate into ectoderm, mesoderm and endoderm. The ES cells still exhibit markers indicative of ES cell status, such as expression of the nuclear transcription factor Oct4, which is associated with pluripotency. Throughout the process and at its end, the human ES cells retain stable karyotypes.

Preferably, at least 90% of the cells in the culture retain all the characteristics of human ES cells described above after at least six passages.

### Example 1

In the first experiments described here human ES cells were plated on irradiated (35 gray gamma irradiation) mouse embryonic fibroblasts. Culture medium for the present work consisted of 80% KNOCKOUT™ Dulbeco's modified Eagle's medium (DMEM) (Gibco BRL, Rockville, Md.), 1 mM L-Glutamine, 0.1 mM β-mercaptoethanol, and 1% nonessential amino acids stock (Gibco BRL, Rockville, Md.), supplemented with either 20% fetal bovine serum (HyClone, Logan, Utah) or 20% KNOCKOUT™ serum replacement (SR), a serum-free replacement originally optimized for mouse ES cells (Gibco BRL, Rockville, Md.). The components of KNOCKOUT™ SR are those described for serum replacements in WO 98/30679.

In alternative experiments medium was supplemented with either serum or the aforesaid serum replacer KNOCKOUT™ SR, and either with or without human recombinant basic fibroblast growth factor (bFGF, 4 ng/ml). The preferred concentration range of bFGF in the culture was between 0.1 ng/ml to 500 ng/ml.

To determine cloning efficiency under varying culture conditions, H-9 cultures were dissociated to single cells for 7 minutes with 0.05% trypsin/0.25% EDTA, washed by centrifugation, and plated on mitotically inactivated mouse embryonic fibroblasts (10⁵ ES cells per well of a 6-well plate). To confirm growth from single cells for the derivation of clonal ES cell lines, individual cells were selected by direct observation under a stereomicroscope and transferred by micropipette to individual wells of a 96 well plate containing mouse embryonic fibroblasts feeders with medium containing 20% serum replacer and 4 ng/ml bFGF.

Clones were expanded by routine passage every 5-7 days with 1 mg/ml collagenase type IV (Gibco BRL, Rockville, Md.). Six months after derivation, H9 cells exhibited a normal XX karyotype by standard G-banding techniques (20 chromosomal spreads analyzed). However, seven months after derivation, in a single karyotype preparation, 16/20 chromosomal spreads exhibited a normal XX karyotype, but 4/20 spreads demonstrated random abnormalities, including one with a translocation to chromosome 13 short arm, one with an inverted chromosome 20, one with a translocation to the number 4 short arm, and one with multiple fragmentation. Subsequently, at 8, 10, and 12.75 months after derivation, H9 cells exhibited normal karyotypes in all 20 chromosomal spreads examined.

We observed that the cloning efficiency of human ES cells in previously described culture conditions that included animal serum was poor (regardless of the presence or absence of bFGF). We also observed that in the absence of animal serum the cloning efficiency increased, and increased even more with bFGF. It has now been established that the addition of FGF facilitated the cultivation of human ES cells in general and is of particular help in facilitating the cloning of human ES cultures.

The data expressed below are the total number of colonies resulting from 10⁵ individualized ES cells plated, +/− standard error of the mean (percent colony cloning efficiency). With 20% fetal serum and no bFGF there was a result of 240+/−28. With 20% serum and bFGF (at 4 ng/ml) the result was about the same, 260+/−12. In the absence of the serum (presence of 20% serum replacer) the result with no bFGF was 633+/−43 and the result with bFGF was 826+/−61. Thus, serum adversely affected cloning efficiency, and the presence of the bFGF in the absence of serum had an added synergistic benefit for cloning efficiency.

The long term culture of human ES cells in the presence of serum does not require the addition of exogenously supplied bFGF, and (as noted above) the addition of bFGF to serum-containing medium does not significantly increase human ES cell cloning efficiency. However, in serum-free medium, bFGF increased the initial cloning efficiency of human ES cells.

Further, it has been discovered that supplying exogenous bFGF is very important for continued undifferentiated proliferation of primate embryonic stem cells in the absence of animal serum. In serum-free medium lacking exogenous bFGF, human ES cells uniformly differentiated by two weeks of culture. Addition of other factors such as LIF (in the absence of bFGF) did not prevent the differentiation.

The results perceived are particularly applicable to clonal lines. In this regard, clones for expansion were selected by placing cells individually into wells of a 96 well plate under direct microscopic observation. Of 192H-9 cells plated into wells of 96 well plates, two clones were successfully expanded (H-9.1 and H-9.2). Both of these clones were subsequently cultured continuously in media supplemented with serum replacer and bFGF.

H9.1 and H9.2 cells both maintained a normal XX karyotype even after more than 8 months of continuous culture after cloning. The H-9.1 and H-9.2 clones maintained the potential to form derivatives of all three embryonic germ layers even after long term culture in serum-free medium. After 6 months of culture, H9.1 and H9.2 clones were confirmed to have normal karyotypes and were then injected into SCID-beige mice.

Both H9.1 and H9.2 cells formed teratomas that contained derivatives of all three embryonic germ layers including gut epithelium (endoderm) embryonic kidney, striated muscle, smooth muscle, bone, cartilage (mesoderm), and neural tissue (ectoderm). The range of differentiation observed within the teratomas of the high passage H9.1 and H9.2 cells was comparable to that observed in teratomas formed by low passage parental H9 cells.

It should be appreciated from the description above that while animal serum is supportive of growth it is a complex mixture that can contain compounds both beneficial and detrimental to human ES cell culture. Moreover, different serum batches vary widely in their ability to support vigorous undifferentiated proliferation of human ES cells. Replacing serum with a clearly defined component reduces the variability of results associated with this serum batch variation, and should allow more carefully defined differentiation studies.

Further, the lower cloning efficiency in medium containing serum suggests the presence of compounds in conventionally used serum that are detrimental to stem cell survival, particularly when the cells are dispersed to single cells. Avoiding the use of these compounds is therefore highly desired.

Feeder Independent Culture

Additional investigations later were directed to the culture of ES cell lines in higher concentrations of FGF but in the absence of both serum and feeder cells. Three different medium formulations have been used in this work, and those medium formulations are referred to here as UM100, BM+ and DHEM. The nomenclature UM100 refers to unconditioned medium to which has been added 100 ng/ml of bFGF. The UM100 medium does contain the Gibco KNOCKOUT™ SR product but does not include or require the use of fibroblast feeder cells of any kind. The BM+ medium is basal medium (DMEM/F12) plus additives, described below, that also permits the culture of cells without feeder cells, but this medium omits the serum replacer product. Lastly, the name DHEM refers to a defined human embryonic stem cell medium. This medium, also described below, is sufficient for the culture, cloning and indefinite proliferation of human ES cells while being composed entirely of inorganic constituents and only human proteins, as opposed to the BM+ medium which contains bovine albumin.

Culture of human ES cells lines H1 and H9 in UM100/BM+/DHEM

UM100 media was prepared as follows: unconditioned media (UM) consisted of 80% (v/v) DMEM/F12 (Gibco/Invitrogen) and 20% (v/v) KNOCKOUT™ SR (Gibco/Invitrogen) supplemented with 1 mM glutamine (Gibco/Invitrogen), 0.1 mM β-mercaptoethanol (Sigma St. Louis, Mo.), and 1% nonessential amino acid stock (Gibco/Invitrogen). To complete the media 100 ng/ml bFGF was added and the medium was filtered through a 0.22 μM nylon filter (Nalgene).

BM+ medium was prepared as follows: 16.5 mg/ml BSA (Sigma), 196 μg/ml Insulin (Sigma), 108 μg/ml Transferrin (Sigma), 100 ng/ml bFGF, 1 mM glutamine (Gibco/Invitrogen), 0.1 mM β-mercaptoethanol (Sigma), and 1% nonessential amino acid stock (Gibco/Invitrogen) were combined in DMEM/F12 (Gibco/Invitrogen) and the osmolality was adjusted to 340 mOsm with 5M NaCl. The medium was then filtered through a 0.22 uM nylon filter (Nalgene).

DHEM medium was prepared as follows: 16.5 mg/ml HSA (Sigma), 196 μg/ml Insulin (Sigma), 108 μg/ml Transferrin (Sigma), 100 ng/ml bFGF, 1 mM glutamine (Gibco/Invitrogen), 0.1 mM β-mercaptoethanol (Sigma), 1% nonessential amino acid stock (Gibco/Invitrogen), vitamin supplements (Sigma), trace minerals (Cell-gro®), and 0.014 mg/L to 0.07 mg/L selenium (Sigma), were combined in DMEM/F12 (Gibco/Invitrogen) and the osmolarity was adjusted to 340 mOsm with 5M NaCl. It is noted that the vitamin supplements in the medium may include thiamine (6.6 g/L), reduced glutathione (2 mg/L) and ascorbic acid PO₄. Also, the trace minerals used in the medium are a combination of Trace Elements B (Cell-gro®, Cat #: MT 99-175-Cl and C (Cell-gro®, Cat #: MT 99-176-Cl); each of which is sold as a 1,000× solution. It is well known in the art that Trace Elements B and C contain the same composition as Cleveland's Trace Element I and II, respectively. (See Cleveland, W. L., Wood, I. Erlanger, B. F., J. Imm. Methods 56: 221-234, 1983.) The medium was then filtered through a 0.22 uM nylon filter (Nalgene). Finally, sterile, defined lipids (Gibco/Invitrogen) were added to complete the medium.

H1 or H9 human embryonic stem cells previously growing on MEF (mouse embryonic fibroblast) feeder cells were mechanically passaged with dispase (1 mg/ml) and plated onto Matrigel™ (Becton Dickinson, Bedford, Mass.). Appropriate medium was changed daily until cell density was determined to be adequate for cell passage. Cells were then passaged with dispase as described and maintained on Matrigel™ (Becton Dickinson).

Growth Rates

To determine the growth rate of human ES cells in the various media, cells were plated at a density of about 5×10⁵ cells/well in triplicate in 6-well tissue culture dishes (Nalgene). On days 3, 5, and 7 the triplicate wells were treated with trypsin/EDTA (Gibco/Invitrogen), individualized and cell numbers were counted. On day 7, additional wells were treated with trypsin, counted, and used to re-seed a new plate at a cell density of about 2×10⁵ cells/well. The day 7

Attachment Dynamics

To determine the attachment rate of human ES cells in the various media cells were plated at a density of 2×10⁵ cells/well in a 6-well tissue culture dish (Nalgene). At time points ranging from 30 minutes to 48 hours unattached cells were washed away and attached cells were removed with trypsin/EDTA (Gibco/Invitrogen) and counted. These experiments were performed to examine if the UM100 growth rate data was due to a combination of better cell attachment and slower growth as opposed to equivalent growth rates for UM100 and CM. We found that attachment percentages were equivalent for both media at all time points tested. Thus, they grow at the same rate.

FACS Analysis of Human ES Cells

Human ES cells were removed from a 6-well tissue culture plate (Nalgene) with trypsin/EDTA (Gibco/Invitrogen)+2% chick serum (ICN Biomedicals, Inc., Aurora, Ohio) for 10 min. at 37° C. The cells were diluted in an equal volume of FACS Buffer (PBS+2% FBS+0.1% Sodium Azide) and filtered through an 80 μM cell strainer (Nalgene). Pellets were collected for 5 min. at 1000 RPM and resuspended in 1 ml 0.5% paraformaldehyde. Human ES cells were fixed for 10 min. at 37° C. and the pellets were collected as described. The ES cells were resuspended in 2 ml FACS Buffer and total cell number was counted with a hemacytometer. Cells were pelleted as described and permeablized for 30 min. on ice in 90% methanol. Human ES cells were pelleted as described and 1×10⁵ cells were diluted into 1 ml of FACS Buffer+0.1% Triton X-100 (Sigma) in a FACS tube (Becton Dickinson). Human ES cells were pelleted as described and resuspended in 50 μl of primary antibody diluted in FACS Buffer+0.1% Triton X-100 (Sigma). Samples of appropriate control antibodies were applied in parallel. Human ES cells were incubated overnight at 4° C. Supernatants were poured off and cells were incubated in the dark for 30 min. at room temperature in 50 μl of secondary antibody (Molecular Probes/Invitrogen). FACS analysis was performed in a Facscalibur™ (Becton Dickinson) cell sorter with CellQuest™ Software (Becton Dickinson). This method for performing FACS analysis allows one to detect cell surface markers, to thus show that you have ES cells. The result observed was that human ES cells cultured in UM100 were 90% positive for Oct-4 as a population. This is comparable to CM-cultured ES cells and confirms that the cells are an ES cell population. For the analysis of SSEA4 and Tra1-60, the process was performed as for Oct-4, except that the cells were not treated in paraformaldehyde or methanol. After cell staining, the cells were re-suspended in FACS buffer (without Triton) and analyzed as described with appropriate antibodies in FACS buffer, again without Triton. The undifferentiated ES cell cultures averaged about 90% positive for these two cell surface markers as well. This was demonstrated by FACS analysis discussed above.

Results

Cells of human ES cell line H1 have now been cultivated in the UM100 medium for over 33 passages (over 164 population doublings) while retaining the morphology and characteristics of human ES cells. H1 cells were cultivated in the BM+ medium for over 6 passages (70 days) while retaining the morphology and characteristics of human ES cells. H9 cells have been cultivated in DHEM medium for over 5 passages (67 days). H9 and H7 human ES cells were also cultivated in UM100 medium in an undifferentiated state for 22 passages and 21 passages respectively. Subsequent testing of the BM+ and UM100-cultured cells established normal karyotypes.

Study of Forms of FGF

Human ES cells of line H1 were cultured under standard conditions in conditioned medium for three passages before being switched to the test media. For the test conditions, cells were cultured on conditioned medium for 24 hours (day 0) and then switched to the test media the next day (day 1). Thereafter the cells were cultured in the respective test media. The human ES cell line H9 was also cultured on Matrigel™ in conditioned media for five passages before being switched to the test media in parallel.

The cells were passaged using the following procedures. The cell cultures were grown to suitable densities (which took approximately 7 days) in 6 well tissue culture plates and then the cultures were treated with 1 ml Dipase (1 mg/ml) (Gibco/Invitrogen) for 5-7 minutes at 37° C. The Dipase was then removed and replaced with 2 ml of the appropriate growth medium. Using a 5 ml pipette, the cells were mechanically removed from the tissue culture plate and then dispersed by pipetting. The cells were then pelleted in a clinical centrifuge for 5 minutes at 1000 rpm. The pellet was then re-suspended in an appropriate volume of medium and replated at desired dilution.

The media formulation was consistent other than the selection of FGF added. The base medium was UM100, with the FGF being variable depending on the desired test condition. The following FGF variants were tested, each added to the medium at 100 ng/ml: FGF1 (acidic FGF), FGF1β (isoform of acidic FGF), FGF2 (basic FGF), FGF3, FGF4, FGF5, FGF6, FGF7, FGF8, FGF9, FGF10, FGF16, FGF17, FGF18, FGF19, FGF20. All FGFs were purchased commercially or produced in recombinant hosts.

The competence of the particular FGF form to support human ES cell cultures was judged after each passage. The conditions which were judged to support human ES cell culture supported cultures that proliferated appropriately in an undifferentiated state in culture, independent of feeder cells, could be passaged effectively, and continued to express the human ES cell markers Oct4, SSEA4, and Tra1-60. The conditions which were judged not to support human ES cells in culture gave rise to cultures in which significant differentiation of the cells was apparent by morphological observation, and the cells were unable to proliferate upon colony passage. The FGF variants which supported human ES cell culture were FGF2, FGF4, FGF17 and FGF18. The FGF variants which did not support maintenance of the human ES cells in an undifferentiated state were FGF1, FGF1B, FGF3, FGF5, FGF6, FGF7, FGF8, FGF10, FGF16, FGF19 and FGF20. The results for the medium with FGF9 added were initially on the margin. Upon repeating the procedure, it appears likely that FGF9 supplemented at 100 ng/ml can also support undifferentiated human ES cells in culture.

At the present time, media supplemented with FGF4, FGF17 and FGF20 have supported undifferentiated human ES cell cultures of H1 cells for 8 passages. Similar replicates with FGF4, FGF17, and FGF18 on human ES cell lines H9 and H14 have extended beyond 6 passages.

### Example 2

### Methods and Materials

Media and cell culture. Unconditioned medium (UM) contained 80% DMEM/F12 and 20% KNOCKOUT serum replacement, and was supplemented with 1 mM L-glutamine, 1% Nonessential Amino Acids (all from Invitrogen), and 0.1 mM β-mercaptoethanol (Sigma). Conditioned medium (CM) is prepared by incubating unconditioned medium with mouse embryonic fibroblasts overnight and collecting the medium afterwards, which is then supplemented with 4 ng/ml bFGF and refrigerated to be used within 2 weeks. Human ES cells were cultured on plates coated with Matrigel™ (BD Scientific) in CM or UM with or without either 0.5 μg/ml mouse noggin (R&D Systems), or 40 ng/ml human bFGF (Invitrogen), or both, and propagated by using 2 mg/ml Dispase (Invitrogen) to loosen the cell colonies. For evaluation of Oct4⁺ cell number, suspended colonies containing 35,000 cells were added to each medium in multiple wells and cultured for 7 days. Cells were harvested and counted on days 1 and 7, and Oct4⁺ cells on day 7 were detected by fluorescence-activated cell sorting (FACS, see below). Embryoid bodies (Ebs) were formed by suspending human ES cells that had been cultured in CM or UM/bFGF/noggin (UMFN), as cell clumps in UM on a non-coated plate, and culturing them on a rocker for 7 days. The EB cells were then re-plated in DMEM medium supplemented with 10% fetal bovine serum on gelatin-coated plate and cultured for 5 days followed by harvesting and reverse transcription-PCR (RT-PCR) analysis. Experiments were repeated multiple times and ANOVA was used for statistic analysis throughout the studies.

Immunoprecipitation and western blotting. 15 ml of DMEM/F12 medium was conditioned on 2.12×10⁵/ml irradiated mouse embryonic fibroblast cells in a T75 flask overnight. The medium was collected and concentrated to about 0.7 ml with a 5 kD molecular weight cut-off filter (Millipore) and immunoprecipiated with goat anti-mouse noggin and gremlin antibodies (R&D Systems) (5 μg each) or 10 μg goat IgG as a negative control. The precipitated proteins or cell lysates (FIG. 2A) were electrophoresized on a 4%-20% linear gradient Polyacrylamide Tris-HCl Precast Gel (BioRad) for western blotting. The antibodies against mouse noggin and gremlin were used for the immunoprecipitated proteins, and antibodies against human Smad1/5/8, phosphorylated Smad1/5/8 (Cell Signaling Technology), BMP2/4 (R&D Systems), and β-Actin (Abcam) were used for the cell lysates. The blots were treated with the ECL substitute solutions 1 and 2 (Amersham Biosciences) and exposed in a Fuji Imager for chemiluminescence.

BMP/Smad-Luciferase Reporter Assay. Human ES cells cultured in CM were transfected with a BMP/Smad-responsive firefly luciferase reporter plasmid, pID120-Lux, together with trace amount of pRL-tk plasmid (Promega) to express Renilla luciferase as an internal control. One day post-transfection, the cells were treated variously for 24 h. Cell lysates were extracted and both the firefly and Renilla luciferase activities tested by using the Dual-Luciferase Reporter Assay System (Promega) on a 3010 Luminometer (BD Biosciences). Results were recorded as the firefly luciferase activity normalized by the Renilla luciferase activity.

Quantitative-PCR and RT-PCR. Total cellular RNA was extracted by RNeasy kit (Qiagen), and treated with RNase-free DNase according to the manufacturer's instructions. One μg RNA was reverse transcribed to cDNA with Improm-II Reverse Transcription System (Promega). Quantitative-PCR was performed by using the SYBR green Q-PCR Mastermix (Stratagene) on the AB 7500 Real Time PCR System (Applied Biosystems) under the following conditions: 10 min at 95° C., 40 cycles of 30 sec at 95° C., 1 min at 60° C., and 1 min at 72° C., and 3 min extension at 72° C. GAPDH transcript was tested as an endogenous reference to calculate the relative expression levels of target genes according to Applied Biosystems' instructions. For RT-PCR, following conditions were used: 3 min at 94° C., various cycles (see below) of 20 sec at 94° C., 30 sec at 55° C., and 1 min at 72° C. The PCR reactions were separated on gel by electrophoresis and the DNA bands were visualized under ultraviolet light for photography. The primer sequences and PCR cycle numbers are listed below.

TABLE 1
Primers and cycle numbers for Q-PCR and RT-PCR

| | | | | PCR |
|---|---|---|---|---|
| Gene | | Forward Primer/ | | Cycle |
| Name | Test | Reverse Primer | SEQ ID NO: | # |
| Id1 | Q-PCR | Forward Primer | | 40 |
| | | 5′-GGT GCG CTG | (SEQ ID NO: | |
| | | TCT GTC TGA G | 1) | |
| | | Reverse Primer | | |
| | | 5′-CTG ATC TCG | (SEQ ID NO: | |
| | | CCG TTG AGG | 2) | |
| Id2 | Q-PCR | Forward Primer | | 40 |
| | | 5′-GCA GCA CCT | (SEQ ID NO: | |
| | | CAT CGA CTA CA | 3) | |
| | | Reverse Primer | | |
| | | 5′-AAT TCA GAA | (SEQ ID NO: | |
| | | GCC TGC AAG GA | 4) | |
| Id3 | Q-PCR | Forward Primer | | 40 |
| | | 5′-CTG GAC GAC | (SEQ ID NO: | |
| | | ATG AAC CAC TG | 5) | |
| | | Reverse Primer | | |
| | | 5′-GTA GTC GAT | (SEQ ID NO: | |
| | | GAC GCG CTG TA | 6) | |
| Id4 | Q-PCR | Forward Primer | | 40 |
| | | 5′-ATG AAG GCG | (SEQ ID NO: | |
| | | GTG AGC CCG GTG | 7) | |
| | | CGC C | | |
| | | Reverse Primer | | |
| | | 5′-TGT GGC CGT | (SEQ ID NO: | |
| | | GCT CGG CCA GGC | 8) | |
| | | AGC G | | |
| GAPDH | Q-PCR | Forward Primer | | 40 |
| | | 5′-GAG TCC ACT | (SEQ ID NO: | |
| | | GGC GTC TTC AC | 9) | |
| | | Reverse Primer | | |
| | | 5′-CTC AGT GTA | (SEQ ID NO: | |
| | | GCC CAG GAT GC | 10) | |
| Oct4 | RT-PCR | Forward Primer | | 35 |
| | | 5′-GGG AAG GTA | (SEQ ID NO: | |
| | | TTC AGC CAA ACG | 11) | |
| | | Reverse Primer | | |
| | | 5′-GGT TCG CTT | (SEQ ID NO: | |
| | | TCT CTT TCG GG | 12) | |
| Nanog | RT-PCR | Forward Primer | | 35 |
| | | 5′-AAT ACC TCA | (SEQ ID NO: | |
| | | GCC TCC AGC AGA | 13) | |
| | | TG | | |
| | | Reverse Primer | | |
| | | 5′-CAA AGC AGC | (SEQ ID NO: | |
| | | CTC CAA GTC ACT | 14) | |
| | | G | | |
| Rex1 | RT-PCR | Forward Primer | | 35 |
| | | 5′-CCT GGA GGA | (SEQ ID NO: | |
| | | ATA CCT GGC ATT | 15) | |
| | | G | | |
| | | Reverse Primer | | |
| | | 5′-TCT GAG GAC | (SEQ ID NO: | |
| | | AAG CGA TTG CG | 16) | |
| CGβ | RT-PCR | Forward Primer | | 30 |
| | | 5′-TGA GAT CAC | (SEQ ID NO: | |
| | | TTC ACC GTG GTC | 17) | |
| | | TCC | | |
| | | Reverse Primer | | |
| | | 5′-TTT ATA CCT | (SEQ ID NO: | |
| | | CGG GGT TGT GGG | 18) | |
| | | G | | |
| Pax6 | RT-PCR | Forward Primer | | 30 |
| | | 5′-CGT CCA TCT | (SEQ ID NO: | |
| | | TTG CTT GGG AAA | 19) | |
| | | TC | | |
| | | Reverse Primer | | |
| | | 5′-GAG CCT CAT | (SEQ ID NO: | |
| | | CTG AAT CTT CTC | 20) | |
| | | CG | | |
| NeuroD1 | RT-PCR | Forward Primer | | 30 |
| | | 5′-AAG CCA TGA | (SEQ ID NO: | |
| | | ACG CAG AGG AGG | 21) | |
| | | ACT | | |
| | | Reverse Primer | | |
| | | 5′-AGC TGT CCA | (SEQ ID NO: | |
| | | TGG TAC CGT AA | 22) | |
| Brachyury | RT-PCR | Forward Primer | | 35 |
| | | 5′-AAC CCA ACT | (SEQ ID NO: | |
| | | GTG GAG ATG ATG | 23) | |
| | | CAG | | |
| | | Reverse Primer | | |
| | | 5′-AGG GGC TTC | (SEQ ID NO: | |
| | | ACT AAT AAC TGG | 24) | |
| | | ACG | | |
| HNF3α | RT-PCR | Forward Primer | | 30 |
| | | 5′-CCA AGC CGC | (SEQ ID NO: | |
| | | CTT ACT CCT ACA | 25) | |
| | | Reverse Primer | | |
| | | 5′-CGC AGA TGA | (SEQ ID NO: | |
| | | AGA CGC TGG AGA | 26) | |
| B-Actin | RT-PCR | Forward Primer | | 25 |
| | | 5′-TGG CAC CAC | (SEQ ID NO: | |
| | | ACC TTC TAC AAT | 27) | |
| | | GAG C | | |
| | | Reverse Primer | | |
| | | 5′-GCA CAG CTT | (SEQ ID NO: | |
| | | CTC CTT AAT GTC | 28) | |
| | | ACG C | | |

FACS and immunocytochemistry. Human ES cells cultured in various media were processed for FACS analysis to detect Oct4⁺ cells. Mouse anti-human Oct4 antibody (Santa Cruz Biotechnology) at 2 μg/ml and fluorescent isothiocyanate-labeled rabbit anti-mouse secondary antibody (Molecular Probes) at 1:1000 dilution were used. Statistic analysis was performed on Arcsine numbers converted from the percentages of Oct4⁺ cells. For immunocytochemistry, the mouse anti-Oct4 antibody (at 0.2 μg/ml) was used and followed by Alexa Fluor 488-labeled anti-mouse IgG secondary antibody (Molecular Probes) at 1:1000 dilution.

Immunoassay of HCG in the culture medium. Human ES cells cultured in UMFN (unconditioned medium with bFGF and noggin) for multiple passages were subsequently cultured in CM plus 100 ng/ml BMP4 up to 7 days with daily refreshment of the medium and BMP4. The spent media were collected on days 3, 5, and 7, and assayed for HCG as described.

G-banding and fluorescence in situ hybridization. Human ES cells cultured in UMFN for various passages were processed for G-banding and fluorescence in situ hybridization. From all the dispersed and fixed cells, 20 cells at metaphase were analyzed for G-banding, and 100-200 nuclei were assayed for fluorescence in situ hybridization using probes to detect marker genes in chromosomes of interest. Representative images captured by the CytoVysion® digital imaging system (Applied Imaging) were reviewed.

Results

UM contains BMP-like differentiation-inducing activity. UM contained 20% KNOCKOUT™ serum replacement (Invitrogen), which includes a proprietary lipid-rich bovine albumin component, ALBUMAX™. UM was conditioned on fibroblasts overnight and then supplemented with 4 ng/ml human bFGF to obtain CM. We cultured human ES cells (H1) in CM, UM, a 1:1 mixture of CM with UM, or a 1:1 mixture of CM with DMEM/F12. The cells in CM or the 1:1 CM-DMEM/F12 mixture remained undifferentiated, and were characterized by typical human ES cell morphology. However, the cells in UM or the 1:1 CM-UM mixture both rapidly differentiated within 48 h. We next substituted purified fetal bovine serum albumin (16.6 g/L, Fisher Scientific) for the serum replacement to determine whether albumin caused the differentiation. This medium allowed human ES cells to maintain an undifferentiated morphology for about 7 days; however, the cells had a reduced proliferation rate and eventually differentiated into a mixed population of cells. These results suggest that components other than albumin contained in the serum replacement are responsible for the rapid differentiation of UM-cultured cells. CM reduces this differentiation-inducing activity, but also provides positive factors to sustain human ES cell self-renewal. In addition to albumin, serum replacement also contains other components that are required for human ES cell culture, so serum replacement rather than albumin was used in all subsequent studies.

To examine whether the differentiation-inducing activity in UM stimulates BMP signaling in human ES cells, we assessed by western blotting the level of phosphorylated Smad1, an immediate effector downstream of BMP receptors. Smad1 phosphorylation (the antibody used here could also detect phosphorylation of other BMP effectors Smad5 and -8) was low in H1 cells cultured in CM, but was high in cells cultured for 24 h in UM, or in CM+BMP4. The addition of noggin to UM reduced the level of Smad1 phosphorylation, but the addition of 40 ng/ml bFGF to UM left the level of Smad1 phosphorylation unchanged. BMP signaling can induce expression of BMP ligands, forming a positive feedback loop in cells from various species, including human ES cells. BMP2/4 proteins were, indeed, detected at an increased level in UM-cultured human ES cells compared to cells cultured in CM or in UM plus noggin. It is at present unclear whether there are BMPs in UM that directly stimulate BMP signaling in human ES cells, or other differentiation-inducing molecules that indirectly stimulate BMP signaling by inducing BMP secretion. Noggin and another BMP antagonist gremlin were both detected in medium conditioned by fibroblasts. These data demonstrate that an elevated, but repressible, BMP signaling activity is present in UM-cultured human ES cells, and that both BMP agonists and antagonists are present in fibroblast-supported culture of human ES cells.

We further assessed BMP signaling in human ES cells (H14) cultured in various media in the presence or absence of protein factors, by using a luciferase reporter plasmid specifically responsive to BMP/Smads. The reporter activity increased with an increasing concentration of the serum replacement or BMP4, and decreased with an increasing concentration of noggin or bFGF. 500 ng/ml Noggin and 40 ng/ml bFGF had synergistic effect in reducing the reporter activity to the level similar to that achieved by CM. Somewhat surprisingly, even higher levels of bFGF (100 ng/ml) reduced BMP signaling to a level comparable to that found in CM without the addition of noggin. These results suggest that serum replacement indeed contains BMP-like activity, which can be reduced by noggin and/or bFGF.

The Id1 promoter contains BMP responsive elements, and Id1 was previously shown to be a target of BMP4 signaling in both human and mouse ESCs. We therefore examined the expression of Id genes as a second indicator of BMP signaling activity in human ES cells cultured in various media. Id1-4 transcripts were higher in human ES cells (H9) cultured for 24 h in UM or CM+BMP4 than in cells cultured in CM, and addition of noggin to UM reduced expression of the Id genes.

UM/bFGF/noggin sustains undifferentiated proliferation of human ES cells UM supplemented with 0.5 μg/ml noggin and 40 ng/ml bFGF sustained undifferentiated proliferation of human ES cells. H1 cells were plated at an equal number and cultured for 7 days in CM, UM, UM plus bFGF, UM plus noggin, or UM plus bFGF and noggin. Oct4⁺ cell numbers were significantly higher after 7 days in CM and UM/bFGF/noggin than in UM, UM/bFGF, or UM/noggin. Intermediate Oct4⁺ cell numbers were detected in UM/bFGF and UM/noggin, suggesting a synergistic effect between noggin and bFGF. Human ES cells cultured in UM/bFGF or UM/noggin could be propagated for multiple passages, but differentiated cells accumulated in either the middle (in UM/bFGF) or edge (in UM/noggin) of the human ES cell colonies. Increased differentiation also occurred in cells cultured in UM/bFGF/noggin if the noggin concentration was reduced to 0.1 μg/ml and the bFGF concentration was reduced to 10 ng/ml. The noggin in UM/bFGF/noggin could be substituted by gremlin (5 μg/ml) or a soluble BMP receptor IA (0.5 μg/ml) (data not shown), supporting that noggin's effects are indeed through the interruption of BMP receptor activation by BMPs.

Three different human ES cell lines (H1, H9, and H14) that had been expanded in UM/bFGF/noggin for more than 40 days (7, 6, and 6 passages, respectively) remained positive for Oct4, but subsequently differentiated if switched to UM lacking bFGF and noggin. UM/bFGF/noggin-cultured human ES cells continued to express other ES cell markers, including Nanog and Rex1, and the cell surface markers SSEA4 and TRA-1-60 (data not shown). Even in the best cultures, human ES cells are mixed with a small percentage of spontaneously differentiated cells. For example, low levels of the trophoblast marker chorionic gonadotropin β-subunit (CGβ) can be detected in CM-cultured ES cells, indicating the existence of small populations of trophoblast. This marker, however, was not detectable in UM/bFGF/noggin-cultured cells. The neural progenitor markers Pax6 and NeuroD1, the mesodermal marker brachyury, and the endodermal marker HNF3α were all negative in CM- and UM/bFGF/noggin-cultured human ES cells. Thus, ES cells propagated in UM/bFGF/noggin maintained characteristic ES cell markers following extended culture.

We further examined human ES cells after long-term culture in UM/bFGF/noggin. H9 cells were continuously cultured in UM/bFGF/noggin for 32 passages. H1 and H14 cells cultured in UM/bFGF/noggin were frozen after passages 20 and 16, respectively. H14 cells were subsequently thawed directly into UM/bFGF/noggin and cultured to passage 18. The population doubling time and percentage of Oct4⁺ cells of both H9 and H14 cells cultured in UM/bFGF/noggin for 27 and 18 passages, respectively, were similar to those for CM-cultured control human ES cells.

UM/bFGF/noggin maintains the developmental potential of human ES cells. When treated with BMP4 in CM for 3-7 days, human ES cells that had been previously cultured in UM/bFGF/noggin for 10 passages differentiated into a flattened epithelium and secreted human chorionic gonadotropin (HCG) into the medium, indicating trophoblast differentiation. Embryoid bodies (EBs) derived from H1 cells cultured in UM/bFGF/noggin for 5 passages, and from control CM-cultured cells, expressed the trophoblast marker CO and markers of the three germ layers, including Pax6, NeuroD1, brachyury, and HNF3α. EB cells also had reduced expression of the ES cell markers Oct4, Nanog, and Rex1. H1 and H9 cells cultured in UM/bFGF/noggin for 7 and 6 passages, respectively, were injected into SCID-beige mice. Teratomas exhibiting complex differentiation developed in the mice 5-6 weeks post-inoculation.

UM/bFGF/noggin-cultured ES cells are karyotypically normal. H1 cells cultured in UM/bFGF/noggin for 5 passages, H9 for 33 passages, and H14 for 19 passages were karyotyped by standard G-banding, and chromosomes 12 and 17 were examined by fluorescence in situ hybridization. The cells retained normal karyotypes.

ES Cells Cultured in Defined and Humanized System Remain Undifferentiated. Although replacement of the CM with UMFN has eliminated the need for mouse-derived feeder cells, the UM still contained fetal bovine serum-derived albumin extract—an incompletely defined component, and the plate-coating material Matrigel™ is a solubilized basement membrane matrix extracted from a mouse tumor. Thus, further removing these animal materials was thought to be appropriate to define a humanized culture system for human ES cells. We first searched for a defined and humanized serum replacement to substitute for the KNOCKOUT SR product previously used, and Sigma's 50× Seral Replacement 3 (SR3) which is composed of three human proteins: albumin, insulin, and transferrin was considered. It has been shown that laminin can substitute for Matrigel™ to coat plates for human ES cell culture in the CM. We then established a system where human ES cells were cultured on laminin-coated plates and in a UM containing 5×SR3 instead of KNOCKOUT SR, plus 40 ng/ml FGF2 and 0.5 μg/ml noggin. ES cells in this system also retained ES cell identity after multiple weekly passages. Therefore, this combination makes up a defined and humanized culture system suitable for human ES cells.

These sets of data, taken together, demonstrate that feeder cells and conditioned medium can be avoided by the use of culture conditions including high concentration of FGF. The other constituents of the culture medium can then be selected to avoid animal products. The result is a highly defined medium that permits the long term culture and proliferation of human embryonic stem cells while retaining all of the potential of those cells.

The present invention has been described above with respect to its preferred embodiments. Other forms of this concept are also intended to be within the scope of the claims. For example, while recombinantly produced human basic fibroblast growth factor was used in the above experiments, naturally isolated fibroblast growth factor should also be suitable. In addition, fibroblast growth factor from many species could also be suitable, due to the high degree of conservation of FGFs between species. Further, these techniques should also prove suitable for use on monkey and other primate cell cultures.

Thus, the claims should be looked to in order to judge the full scope of the invention.

### INDUSTRIAL APPLICABILITY

The present invention provides methods for culturing primate embryonic stem cells, and culture media for use therewith.

We claim:

1. A method of culturing human embryonic stem cells, comprising:
culturing human stem cells on a matrix in a culture medium free of serum and in a stem cell culture medium containing amino acids, vitamins, salts, minerals, transferrin or a transferrin substitute, insulin or an insulin substitute, albumin and a fibroblast growth factor supplied from a source other than a feeder layer, the fibroblast growth factor present in a concentration at least as high as a maintenance concentration, wherein the medium supports the culture and proliferation of undifferentiated proliferating euploid human embryonic stem cells for at least six passages without feeder cells or conditioned medium, wherein at least 90% of the cells in the culture remain undifferentiated.

2. The method of claim 1 wherein the FGF is selected from FGF2, FGF4, FGF17 and FGF18.

3. The method of claim 1 wherein the FGF is FGF2 which is present in the medium in at least 40 ng/ml.

4. The method of claim 1 wherein the FGF is FGF2 which is present in the medium in at least 100 ng/ml.

5. A method of culturing human embryonic stem cells in defined media without serum and without feeder cells, the method comprising:
culturing human embryonic stem cells on a matrix in a culture medium containing albumin, amino acids, vitamins, minerals, at least one transferrin or transferrin substitute, at least one insulin or insulin substitute, the culture medium free of serum and containing at least about 100 ng/ml of a fibroblast growth factor, and culturing without feeder cells or conditioned medium, wherein the medium supports proliferation of at least 90% of the human embryonic stem cells in an undifferentiated state.

6. The method of claim 4, wherein said culturing step includes the embryonic stem cells proliferating in culture for over one month while maintaining the potential of the embryonic stem cells to differentiate into derivatives of endoderm, mesoderm, and ectoderm tissues, and while maintaining the karyotype of the embryonic stem cells.

7. The method of claim 4, wherein the FGF is selected from FGF2, FGF4, FGF17 and FGF18.

8. A culture of human embryonic stem cells comprising:
(a) human embryonic stem cells;
(b) a matrix; and
(c) a stem cell medium containing albumin, amino acids, vitamins, minerals, at least one transferrin or transferrin substitute, at least one insulin or insulin substitute, the culture medium free of serum and containing a fibroblast growth factor supplied from a source other than a feeder layer, the fibroblast growth factor present in a concentration at least as high as a maintenance concentration, wherein the medium supports the culture of the embryonic stem cells indefinitely in the absence of serum and in the absence of feeder cells and also in the absence of medium exposed to feeder cells,
wherein the culture maintains at least 90% of the embryonic stem cells in an undifferentiated state indefinitely with stable karyotype.

9. The culture of claim 7 wherein the fibroblast growth factor is FGF2 which is present in the medium in a concentration of at least about 100 ng/ml.

10. A culture of feeder independent human embryonic stem cells comprising:
human embryonic stem cells on a matrix in a stem cell culture medium, the stem cell culture medium comprising albumin, amino acids, vitamins, minerals, at least one transferrin or transferrin substitute, at least one insulin or insulin substitute, the culture medium free of serum and containing a fibroblast growth factor present in a concentration at least as high as a maintenance concentration, wherein the fibroblast growth factor is selected from FGF2, FGF4, FGF17, and FGF18, wherein the culture is independent of feeder cells while at least 90% of the human embryonic stem cells remain euploid and in an undifferentiated state.

11. A culture as claimed in claim 9, wherein the fibroblast growth factor is present at a concentration of at least about 100 ng/ml.

12. A culture as claimed in claim 9, wherein the human embryonic stem cells remain euploid and in a undifferentiated state for at least six passages.
